(19) United States
(12) Patent Application Publication
(10) Pub. No.: US 2026/0112027 A1
(43) Pub. Date: Apr. 23, 2026

(54) ADAPTIVE MACHINE LEARNING-BASED LESION IDENTIFICATION

(71) Applicant: The Regents of the University of Colorado, a body corporate, Denver, CO (US)

(72) Inventors: Bennett CHIN, Denver, CO (US); Michael Stephen SILOSKY, Denver, CO (US); Fuyong XING, Denver, CO (US); Xinyi YANG, Denver, CO (US)

(21) Appl. No.: 19/120,560

(22) PCT Filed: Oct. 11, 2023
(86) PCT No.: PCT/US2023/076590

§ 371 Date: Apr. 11, 2025

Related U.S. Application Data
(60) Provisional application No. 63/379,131, filed on Oct. 11, 2022.

(51) Int. Cl. G06T 7/00; G16H 30/40

(52) U.S. Cl. CPC G06T 7/0012; G16H 30/40; G06T 2207/10104; G06T 2207/20081; G06T 2207/30096

(57) ABSTRACT

An adaptable deep learning method is provided that delivers sound hepatic lesion identification in NETs, while significantly reducing human effort for data annotation and improving model generalizability for PET image quantification. A region-guided GAN (RGGAN) model conducts image-to-image translation between list-mode simulated PET images and real-world clinical data, while preserving semantic content of interest, e.g., lesions. The RG-GAN model is integrated with a lesion detection model into an end-to-end, unified framework for joint-task learning, such that the two models can benefit from each other. The RG-GAN translates the list-mode simulated data into real world-style images, which appear to be drawn from the real clinical PET image dataset, and feeds the translated images into the lesion detection model for training. In order to deal with the limited diversity of list mode-simulated PET image data, a specific data augmentation module is incorporated into the unified framework to improve model training.

### CROSS-REFERENCE TO RELATED APPLICATION

This application claims priority to U.S. Provisional Application No. 63/379,131, titled “ADAPTIVE MACHINE LEARNING-BASED LESION IDENTIFICATION.” filed on Oct. 11, 2022, the entire disclosure of which is hereby incorporated by reference in its entirety.

### BACKGROUND

Lesion detection with positron emission tomography (PET) imaging is important for tumor staging, treatment planning, and advancing novel therapies to improve patient outcomes. Deep neural networks have been recently adopted to identify glycolytically active lesions in fluorodeoxyglucose (FDG) PET. However, current deep learning methods typically rely on a large amount of well-annotated data for model training. This is extremely difficult to achieve for neuroendocrine tumors (NETs), because of a low incidence of NETs and expensive lesion annotation in PET images.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

According to aspects of the present disclosure, an adaptable deep learning method has been designed and trained for hepatic lesion detection in real-world clinical NET PET images without the need for real lesion annotated data but instead using low-cost list-mode simulated data.

In particular, a region-guided generative adversarial network (RG-GAN) is proposed for lesion-preserved image translation between list-mode simulated PET images and unannotated real-world data. Then, a specific data augmentation module is proposed for the list-mode simulated data and incorporated into the RG-GAN to improve model training. Then, the RG-GAN, the data augmentation module, and a lesion detection neural network are combined into a unified framework for joint task learning to adaptatively identify lesions in real-world data. Testing suggests that the unified framework outperforms other state-of-the-art lesion detection methods in real clinical Ga-DOTATATE PET images and produces very competitive performance with models that are trained with real lesion annotations. Thus, RG-GAN modeling with specific data augmentation can be used to obtain good lesion detection performance without using real data annotations. The disclosed adaptable deep learning method delivers sound hepatic lesion identification in NETs, while significantly reducing human effort for data annotation and improving model generalizability for PET image quantification.

This Summary is provided to introduce a selection of concepts in a simplified form, which is further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 illustrates a diagram of a unified framework for adaptive machine learning-based lesion identification, according to aspects described herein.

FIG. 2 illustrates different methods of lesion detection, according to aspects described herein.

FIG. 3 illustrates an example of a suitable operating environment in which one or more aspects of the present application may be implemented.

### DETAILED DESCRIPTION

Various aspects of the disclosure are described more fully below with reference to the accompanying Appendix, which forms a part hereof, and which shows specific example aspects. However, different aspects of the disclosure may be implemented in many different ways and should not be construed as limited to the aspects set forth herein. The following detailed description is, therefore, not to be taken in a limiting sense.

The present disclosure provides an adaptable deep learning method that delivers sound hepatic lesion identification in NETs, while significantly reducing human effort for data annotation and improving model generalizability for PET image quantification. A region-guided GAN (RGGAN) model is proposed to conduct image-to-image translation between list-mode simulated PET images and real-world clinical data. The RG-GAN model can preserve semantic content of interest. e.g., lesions, during image translation so that the lesions in the translated simulated PET images can be used as a supervision signal in domain adaptation for lesion detection. The RG-GAN model is integrated with a lesion detection model into an end-to-end, unified framework for joint-task learning, such that the two models can benefit from each other. The RG-GAN translates the list-mode simulated data into real world-style images, which appear to be drawn from the real clinical PET image dataset, and feeds the translated images into the lesion detection model for training. The lesion detection model sends feedback to the RG-GAN and assists with its training to produce proper translated images. In order to deal with the limited diversity of list mode-simulated PET image data (as these images are synthesized from only one real, healthy subject), a specific data augmentation module is incorporated into the unified framework to improve model training. In this way, a state-of-the-art deep learning model to detect lesions in real clinical PET images is provided without using any real data annotations.

As an example, aspects of the adaptive deep learning model include at least the following:

(1) A hepatic lesion detection model using list mode-simulated PET images and apply it to real-world PET data. With list-mode reconstruction, a large set of liver PET images can be generated to train a deep learning model without real data annotations for hepatic lesion detection.

(2) An RG-GAN model for image-to-image translation is combined with a lesion detection model into a unified framework for unsupervised domain adaptation. Compared with other GAN neural networks, the disclosed RG-GAN explicitly highlights the lesion regions so that the lesions are preserved during image translation. This aids lesion identification in PET images, where lesions are often difficult to detect due to high image noise, low spatial resolution and small object size. Thus, the RG-GAN facilitates learning of the lesion detection model in the unified framework and enables unsupervised domain adaptation between list mode-simulated PET images and unannotated real clinical data.

(3) A specific data augmentation pipeline to enhance model training within the unified framework. Since the list-mode simulated PET images have limited diversity, e.g., spherical-shaped lesions and similar image background, a series of geometric and visual image transformations are designed and applied for model training, such that the RG-GAN produces more realistic PET images for the lesion detection model.

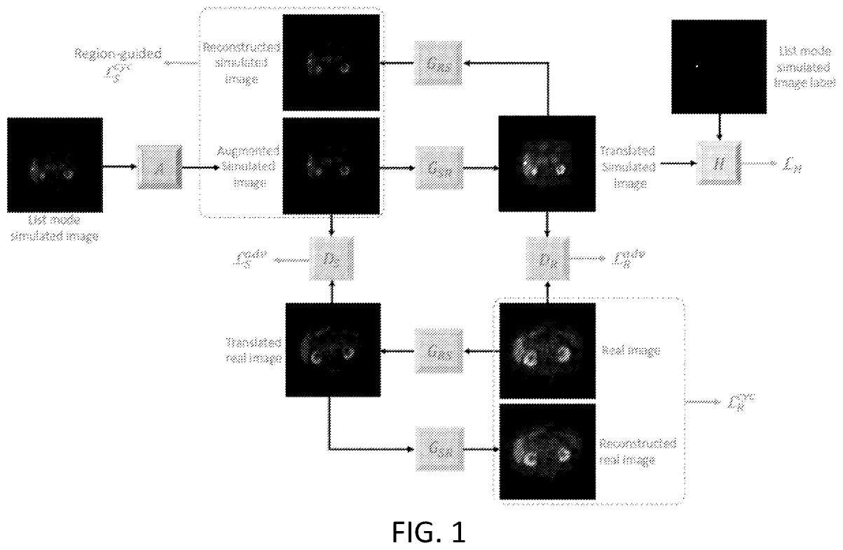

FIG. 1 illustrates a diagram of a unified framework for adaptive machine learning-based lesion identification. As illustrated, GSR is the simulated-to-real generator to transform list-mode simulated images to real world-style PET images, and DR is the corresponding discriminator to classify whether an input image is real. GRS and DS are the real-to-simulated generator and its associated discriminator, respectively. As depicted in FIG. 1, “A” denotes the specifically designed data augmentation module for the list-mode simulated image data, and “H” represents the lesion detection model, which is trained with translated simulated images and associated image labels.

As illustrated, X_{S} denotes the list-mode simulated training PET image data and Y_{S} represent the associated labels, each of which is a 3D binary image for one synthesized subject, with, for example, 1's for lesions and 0's for the other regions. As shown, X_{R} denotes the unannotated real-world clinical PET image data. Specifically, the RG-GAN may include two generator-discriminator pairs, G_{SR}-D_{R} and G_{RS}-D_{S} for simulated-to-real and real-to-simulated image translation, respectively. The generator G_{SR} learns to convert simulated images X_{S} to real world-style data G_{SR}(X_{S}) such that the corresponding discriminator D_{R} cannot distinguish the translated simulated images G_{SR}(X_{S}) from the real ones X_{R}. Similarly, the real-to-simulated generator G_{RS} conducts image translation in the reversed direction, trying to fool its associated discriminator D_{S}. An image reconstruction-based cycle-consistency constraint is used to enable unpaired image translation between list-mode simulated and real-world data. A lesion-specific, weighted matrix is designed and applied to the image reconstruction to preserve lesion regions during image translation.

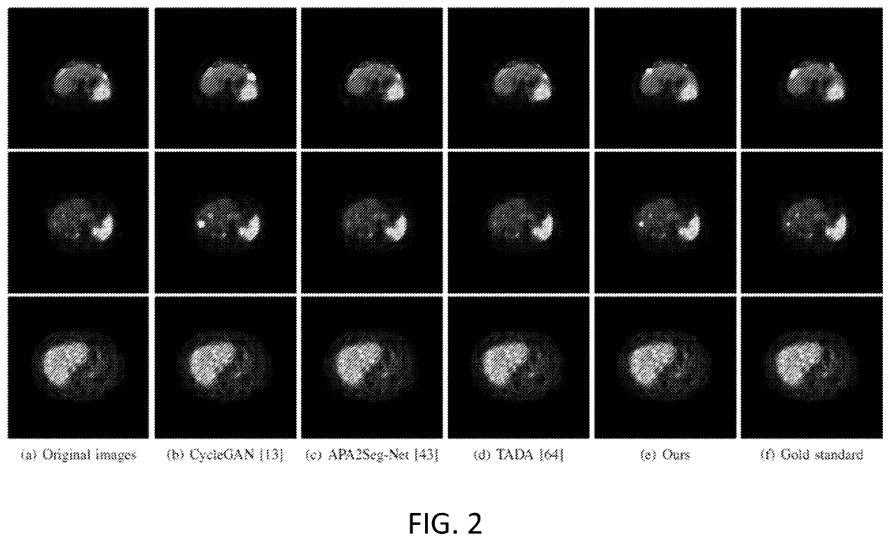

FIG. 2 illustrates qualitative results of different methods of lesion detection. In particular, rows 1 and 2 represent two different subjects with lesions (i.e., regions marked with different colors) and row 3 denotes a normal subject without lesions.

TABLE I
COMPARISON WITH STATE-OF-THE-ART UDA METHODS IN
TERMS OF PRECISION, RECALL, AND F₁ SCORE (%).

| Models | Precision | Recall | F₁ score |
|---|---|---|---|
| CycleGAN [13] | 76.1 | 55.9 | 64.4 |
| APA2Seg-Net [43] | 48.0 | 49.0 | 48.5 |
| TADA [64] | 78.6 | 58.3 | 66.9 |
| Ours | 80.7 | 67.9 | 73.8 |

Table I presents the comparison results between the disclosed method and several other UDA segmentation approaches, including anatomy-preserving domain adaptation to segmentation network (APA2Seg-Net) and tumor-aware adversarial domain adaptation (TADA). A CycleGAN is used to translate the list-mode simulated PET images to real world-style data, which translated data is then used to train a deep neural network for lesion detection. Table I presents the comparison results for these methods in terms of precision, recall, and F1 score. The disclosed method outperforms the others by a large margin, from about 6.9% to about 25.3% in the F1 score. Specifically, CycleGAN gives a low FT score probably due to no mechanism to encourage semantic content preservation during image translation. TADA produces relatively better performance than CycleGAN, perhaps because they incorporate the labels of source data into model training and help maintain geometric structure in input images. APA2Seg-Net provides a surprisingly low F1 score, probably because the general-purpose MIND and correlation coefficient regularization may not be applicable to the challenging PET image data used in this study. The disclosed method produces the best performance, demonstrating its effectiveness to deal with PET-image UDA.

TABLE II
ABLATION STUDY, ISO., ANISO, AND NOISE REPRESENT
RANDOM ISOTROPIC SCALING, ANISOTROPIC SCALING
AND NOISE CORRUPTION, RESPECTIVELY.

| Models | Precision | Recall | F₁ score |
|---|---|---|---|
| Simulated | 85.0 | 27.2 | 41.3 |
| Baseline | 50.3 | 62.1 | 55.6 |
| Data-Augment | 67.5 | 65.9 | 66.7 |
| RG-GAN | 79.1 | 61.4 | 69.1 |
| RG-GAN + iso. | 76.5 | 64.1 | 69.8 |
| RG-GAN + iso. + aniso. | 73.2 | 68.6 | 70.8 |
| RG-GAN + iso. + aniso. + noise | 81.9 | 64.1 | 72.0 |
| Ours | 80.7 | 67.9 | 73.8 |
| Real | 88.9 | 71.8 | 79.4 |

Table II shows the results of an ablation study to evaluate each component of the disclosed method. The different components include: 1) Simulated: Train a lesion detection model using only the list-mode simulated PET image data and directly apply this model to lesion detection in real-world data. 2) Baseline: The disclosed method without weighted image reconstruction or the specific data augmentation module. 3) Data-Augment: The disclosed method without the weighted image reconstruction but with the specific data augmentation. 4) RG-GAN: The disclosed method with the weighted image reconstruction but without the specific data augmentation.

To evaluate the effectiveness of each type of image transformation in the specific stochastic data augmentation module, some variants of the disclosed method were trained by sequentially adding one transformation: 5) only random isotropic scaling (RG-GAN+iso:):6) random isotropic scaling and anisotropic scaling (RG-GAN+iso:+aniso:); 7) random isotropic scaling, anisotropic scaling and noise corruption (RG-GAN+iso:+aniso:+noise), and 8) using all the image transformations (Ours). The Real means training a lesion detection model with real-world data annotations and testing on real-world data.

The Simulated model produces the lowest F1 score, which is because it does not address the domain shift between the list-mode simulated and real-world data. The Baseline model improves the performance to an F1 score of 55.6%, but it is much lower than the result of the Data-Augment or RG-GAN model. This demonstrates the effectiveness of the proposed list-mode specific data augmentation and weighted image reconstruction. With a combination of the specific data augmentation and the RG-GAN, the lesion detection performance can be further boosted. In particular, when using all the designed image transformations, the F1 score can be further increased to 73.8%, thereby greatly reducing the gap to the fully supervised Real model that is directly trained with real-world PET image annotations. This suggests that the proposed data augmentation module is beneficial to UDA model training for PET images.

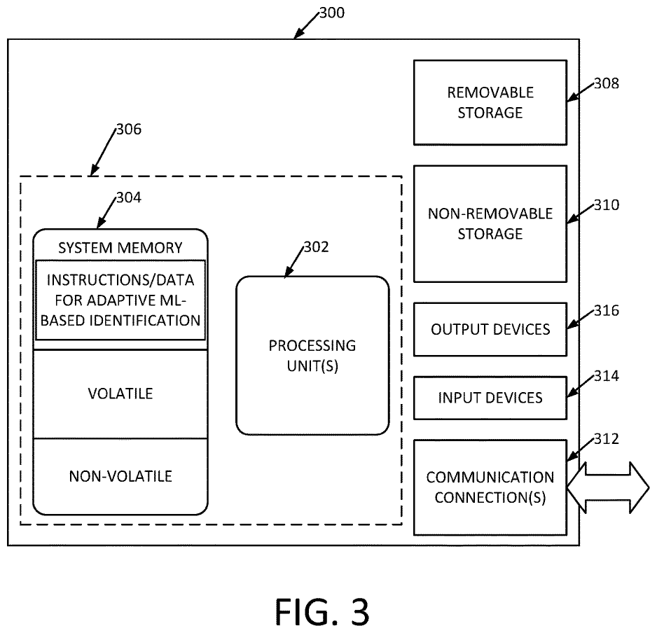

FIG. 3 illustrates an example of a suitable operating environment 300 in which one or more of the present embodiments may be implemented. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smart phones, network PCs, minicomputers, mainframe computers, distributed computing environments that include any of the above systems or devices, and the like.

In its most basic configuration, operating environment 300 typically may include at least one processing unit 302 and memory 304. Depending on the exact configuration and type of computing device, memory 304 (storing, among other things, APIs, programs, etc. and/or other components or instructions to implement or perform the system and methods disclosed herein, etc.) may be volatile (such as RAM), non-volatile (such as ROM, flash memory, etc.), or some combination of the two. This most basic configuration is illustrated in FIG. 3 by dashed line 306. Further, environment 300 may also include storage devices (removable, 308, and/or non-removable, 310) including, but not limited to, magnetic or optical disks or tape. Similarly, environment 300 may also have input device(s) 314 such as a keyboard, mouse, pen, voice input, etc. and/or output device(s) 316 such as a display, speakers, printer, etc. Also included in the environment may be one or more communication connections, 312, such as LAN, WAN, point to point, etc.

Operating environment 300 may include at least some form of computer readable media. The computer readable media may be any available media that can be accessed by processing unit 302 or other devices comprising the operating environment. For example, the computer readable media may include computer storage media and communication media. The computer storage media may include volatile and nonvolatile, removable and non-removable media implemented in any method or technology for storage of information such as computer readable instructions, data structures, program modules or other data. The computer storage media may include RAM, ROM, EEPROM, flash memory or other memory technology, CDROM, digital versatile disks (DVD) or other optical storage, magnetic cassettes, magnetic tape, magnetic disk storage or other magnetic storage devices, or any other non-transitory medium, which can be used to store the desired information. The computer storage media may not include communication media.

The communication media may embody computer readable instructions, data structures, program modules, or other data in a modulated data signal such as a carrier wave or other transport mechanism and includes any information delivery media. The term “modulated data signal” may mean a signal that has one or more of its characteristics set or changed in such a manner as to encode information in the signal. For example, the communication media may include a wired media such as a wired network or direct-wired connection, and wireless media such as acoustic, RF, infrared and other wireless media. Combinations of the any of the above should also be included within the scope of computer readable media.

The operating environment 300 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

The different aspects described herein may be employed using software, hardware, or a combination of software and hardware to implement and perform the systems and methods disclosed herein. Although specific devices have been recited throughout the disclosure as performing specific functions, one skilled in the art will appreciate that these devices are provided for illustrative purposes, and other devices may be employed to perform the functionality disclosed herein without departing from the scope of the disclosure.

As stated above, a number of program modules and data files may be stored in the system memory 304. While executing on the processing unit 302, program modules (e.g., applications, Input/Output (I/O) management, and other utilities) may perform processes including, but not limited to, one or more of the stages of the operational methods described herein such as the methods described herein with respect to FIGS. 1 and 2, for example.

Furthermore, examples of the invention may be practiced in an electrical circuit comprising discrete electronic elements, packaged or integrated electronic chips containing logic gates, a circuit utilizing a microprocessor, or on a single chip containing electronic elements or microprocessors. For example, examples of the invention may be practiced via a system-on-a-chip (SOC) where each or many of the components illustrated in FIG. 3 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality described herein may be operated via application-specific logic integrated with other components of the operating environment 300 on the single integrated circuit (chip). Examples of the present disclosure may also be practiced using other technologies capable of performing logical operations such as, for example, AND, OR, and NOT, including but not limited to mechanical, optical, fluidic, and quantum technologies. In addition, examples of the invention may be practiced within a general purpose computer or in any other circuits or systems.

As will be understood from the foregoing disclosure, one aspect of the technology relates to a system comprising: at least one processor; and memory storing instructions that, when executed by the at least one processor, causes the system to perform a set of operations. The set of operations comprises: obtaining first patient imaging data; generating, based on the first patient imaging data, list-mode simulated positron emission tomography (PET) images; performing an image-to-image translation between the list-mode simulated PET images and real-world clinical data associated with a feature; training a machine learning model to detect the feature using the translated list-mode simulated PET images; obtaining second patient imaging data; processing the second patient imaging data according to the trained machine learning model to generate a model processing result associated with the feature; and providing an indication of the model processing result associated with the feature. In an example, the set of operations further comprises: applying data augmentation to the list-mode simulated PET images, wherein the data augmentation increases diversity of the list-mode simulated PET images. In another example, the translation between the list-mode simulated PET images and the real-world clinical data includes highlighting the feature in the translated list-mode simulated PET images. In a further example, the training is unsupervised. In yet another example, the real-world clinical data in unannotated. In a further still example, the training the machine learning model enables unsupervised domain adaptation between the translated list-mode simulated PET images and the unannotated real-world clinical data. In another example, a translation model is used to perform the image-to-image translation between the list-mode simulated PET images and the real-world clinical data associated with the feature. In a further example, feedback from training the machine learning model is used to train the translation model. In yet another example, highlighting the feature in the translated list-mode simulated PET images provides a supervision signal in domain adaptation for lesion detection.

In another aspect, the technology relates to a method of adaptively training a machine learning model to detect a feature. The method comprises: obtaining first patient imaging data: generating, based on the first patient imaging data, list-mode simulated positron emission tomography (PET) images; performing an image-to-image translation between the list-mode simulated PET images and real-world clinical data associated with the feature, wherein the translation includes highlighting the feature in the translated list-mode simulated PET images; training the machine learning model to detect the feature using the translated list-mode simulated PET images; obtaining second patient imaging data; processing the second patient imaging data according to the trained machine learning model to generate a model processing result associated with the feature; and providing an indication of the model processing result associated with the feature. In an example, the method further comprises applying data augmentation to the list-mode simulated PET images, wherein the data augmentation increases diversity of the list-mode simulated PET images. In another example, the training is unsupervised. In a further example, the real-world clinical data in unannotated. In yet another example, the training the machine learning model enables unsupervised domain adaptation between the translated list-mode simulated PET images and the unannotated real-world clinical data. In a further still example, a translation model is used to perform the image-to-image translation between the list-mode simulated PET images and the real-world clinical data associated with the feature. In yet another example, feedback from training the machine learning model is used to train the translation model. In another example, highlighting the feature in the translated list-mode simulated PET images provides a supervision signal in domain adaptation for lesion detection.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, for example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A system comprising:
at least one processor; and
memory storing instructions that, when executed by the at least one processor, causes the system to perform a set of operations, the set of operations comprising:
obtaining first patient imaging data;
generating, based on the first patient imaging data, list-mode simulated positron emission tomography (PET) images;
performing an image-to-image translation between the list-mode simulated PET images and real-world clinical data associated with a feature;
training a machine learning model to detect the feature using the translated list-mode simulated PET images;
obtaining second patient imaging data;
processing the second patient imaging data according to the trained machine learning model to generate a model processing result associated with the feature; and
providing an indication of the model processing result associated with the feature.

2. The system of claim 1, the set of operations further comprising:
applying data augmentation to the list-mode simulated PET images, wherein the data augmentation increases diversity of the list-mode simulated PET images.

3. The system of claim 1, wherein the translation between the list-mode simulated PET images and the real-world clinical data includes highlighting the feature in the translated list-mode simulated PET images.

4. The system of claim 1, wherein the training of the machine learning model is unsupervised.

5. The system of claim 1, wherein the real-world clinical data is unannotated.

6. The system of claim 5, wherein the training the machine learning model enables unsupervised domain adaptation between the translated list-mode simulated PET images and the unannotated real-world clinical data.

7. The system of claim 1, wherein a translation model is used to perform the image-to-image translation between the list-mode simulated PET images and the real-world clinical data associated with the feature.

8. The system of claim 7, wherein feedback from training the machine learning model is used to train the translation model.

9. The system of claim 3, wherein highlighting the feature in the translated list-mode simulated PET images provides a supervision signal in domain adaptation for lesion detection.

10. A method of adaptively training a machine learning model to detect a feature, the method comprising:
obtaining first patient imaging data;
generating, based on the first patient imaging data, list-mode simulated positron emission tomography (PET) images;
performing an image-to-image translation between the list-mode simulated PET images and real-world clinical data associated with the feature, wherein the translation includes highlighting the feature in the translated list-mode simulated PET images;
training the machine learning model to detect the feature using the translated list-mode simulated PET images;
obtaining second patient imaging data;
processing the second patient imaging data according to the trained machine learning model to generate a model processing result associated with the feature; and
providing an indication of the model processing result associated with the feature.

11. The method of claim 10, further comprising:
applying data augmentation to the list-mode simulated PET images, wherein the data augmentation increases diversity of the list-mode simulated PET images.

12. The method of claim 10, wherein the training of the machine learning model is unsupervised.

13. The method of claim 10, wherein the real-world clinical data is unannotated.

14. The method of claim 13, wherein the training the machine learning model enables unsupervised domain adaptation between the translated list-mode simulated PET images and the unannotated real-world clinical data.

15. The method of claim 10, wherein a translation model is used to perform the image-to-image translation between the list-mode simulated PET images and the real-world clinical data associated with the feature.

16. The method of claim 15, wherein feedback from training the machine learning model is used to train the translation model.

17. The method of claim 10, wherein highlighting the feature in the translated list-mode simulated PET images provides a supervision signal in domain adaptation for lesion detection.

18. A method of adaptively training a machine learning model to detect a feature, the method comprising:
obtaining patient imaging data;
processing the patient imaging data according to a machine learning model to generate a model processing result associated with the feature, wherein the machine learning model was trained to detect the feature using a set of list-mode simulated positron emission tomography images each including highlighting corresponding to the feature based on real-world clinical data associated with the feature; and
providing an indication of the model processing result associated with the feature for the patient imaging data.

19. The method of claim 18, wherein the real-world clinical data is unannotated.

20. The method of claim 18, wherein the highlighting provides a supervision signal in domain adaptation for lesion detection.
